(19) United States
(12) Patent Application Publication
(10) Pub. No.: US 2014/0039636 A1
(43) Pub. Date: Feb. 6, 2014

(54) Connecting Mechanism for Medial and Lateral Polyethylene Bearing Surfaces for Knee Replacement

(71) Applicant: William B. Kurtz, Nashville, TN (US)

(72) Inventor: William B. Kurtz, Nashville, TN (US)

(21) Appl. No.: 13/957,275

(22) Filed: Aug. 1, 2013

Related U.S. Application Data
(60) Provisional application No. 61/678,189, filed on Aug. 1, 2012.

(51) Int. Cl. A61F 2/38

(52) U.S. Cl. CPC A61F 2/389
USPC 623/20.32

(57) ABSTRACT

Disclosed herein are improved methods, apparatus and/or systems for a tibial implant assembly that can facilitate balancing, positioning, insertion, locking and maneuvering of the modular tibial inserts during knee surgery. The system may include a plurality of modular tibial inserts with locking or engagement mechanisms that allow creation of a modular insert assembly, and a corresponding tibial tray and optional tray components. The system allows the quick and convenient mating and locking of the tibial insert assembly to the tibial tray. The various components can accommodate mobile-bearing and fixed-bearing designs for the tibial tray.

### CROSS-REFERENCE TO RELATED APPLICATIONS

This application claims the benefit of U.S. Provisional Patent Application Ser. No. 61/678,189 entitled “Connecting Mechanism for Medial and Lateral Polyethylene Bearing Surfaces for Knee Replacement,” filed Aug. 1, 2012. The disclosure of this document is incorporated by reference in its entirety.

### TECHNICAL FIELD

The invention relates to improved orthopedic implants for use during a joint replacement procedure. More specifically, disclosed herein are improved methods, apparatus, and/or systems for a tibial implant component system that facilitate the adaptability and reliability of the system during surgery, including more accurately restoring normal kinematics in the knee, reducing the risk of implant failure and/or component dislocation and decreasing implant wear.

### BACKGROUND OF THE INVENTION

In a typical knee joint replacement procedure, a surgical implant can include one or more metal, plastic or ceramic “base” components that are bonded or otherwise attached to the various bones of the joint, and such implants typically also include meniscal bearing liners or “inserts” that can be attached to the base components. Such inserts are often provided in multiple sizes and/or shapes (or incorporate other varying characteristics to allow a suitable insert to be selected during the surgical procedure) and attached to the base component, thereby altering the operation and/or performance of the implant in a desired manner. For example, in a knee joint, the knee implant component assembly can include a metal femoral component, a metal tibial tray base component, and a tibial insert (i.e., a meniscal bearing or liner that sits between the two metal components) that may be available in different sizes for the surgical procedure.

Tibial Inserts are a common component of knee implant designs, and may include fixed-bearing inserts as well as mobile-bearing inserts. A fixed-bearing insert is typically securely fixed to the tibial tray and the femoral component rolls, translates and/or otherwise moves relative to the insert articulating surface. In essence, the relative motion at the tibiofemoral junction takes place between the metal femoral component and the articulating surface of the tibial insert. In contrast, a mobile-bearing insert typically moves with respect to the tibial tray in some manner and does not restrict the natural movement of the femoral component as much as a comparable fixed-bearing component.

Recently, manufacturers have been exploring the use of separate medial and lateral inserts that fit into a single tibial tray, which is believed to better accommodate the differing anatomical characteristics of the medial and lateral condyles of the natural knee joint (and corresponding medial and lateral surfaces of the tibial bone). While such designs could potentially improve the ability of the surgeon to “balance” the patient's knee using appropriate combinations of medial and lateral inserts of different shapes, sizes and/or thicknesses that each secure into the tibial tray, the manipulation, positioning and maneuvering of multiple inserts in the knee joint can significantly increase the opportunity for implant wear, implant failure and/or surgical error.

### BRIEF SUMMARY OF THE INVENTION

The invention disclosed herein includes the realization of a need for an improved tibial insert design and associated knee joint implant components that facilitate the balancing of a knee implant using multiple insert components, yet allow the surgeon to easily and effectively connect the multiple insert components to the tibial tray in a single operation. The improved implant component assembly may include tibial insert components that “mate” or can otherwise be assembled by a surgeon outside of the surgical “cavity” (i.e., outside of the knee joint), thereby forming a single assembly or “hybrid” tibial insert component. The single tibial insert assembly can then introduced into the surgical cavity, and secured with the relevant base component (i.e., the tibial tray).

In various embodiments, tibial insert components can include mating features such as extensions and/or docking cavities that allow the medial and lateral tibial insert components to be connected prior to placement onto the tibial tray. In various other embodiments, a separate locking tool or other engagement component can be provided that connects the medial and lateral tibial inserts together prior to placement by the surgeon. In various additional alternative embodiments, the improved implant component assembly may also include a tibial tray design that incorporates either fixed or mobile-bearing features, as desired by the surgeon, to optimize the surgical repair of the knee. Various features of the present invention may be applied to a wide variety of both mobile-bearing and fixed-bearing knee designs, which may desirably include the use of a single tibial insert assembly that incorporates separate medial and lateral tibial insert components selected by the surgeon from insert sets, resulting in knee implants of varying structure, function and design.

In various exemplary embodiments, the knee prosthesis components may include two-piece or separate modular tibial inserts. The tibial insert overall shape and thickness variations (or other structural dissimilarities between inserts of a given set) of the medial and lateral tibial insert components may be designed and manufactured using standard techniques known in the art. The techniques may include dimensions derived from a standard library database and/or from patient-specific images taken from the surgeon.

In various embodiments, the modular tibial inserts may be designed with an integrated frictional or other locking mechanisms, such as a dove-tail and/or lock-and-key mechanisms. The tibial insert locking mechanism can desirably allow the medial and lateral inserts to quickly mate and lock together. For example, a medial tibial insert may be desirably designed with a female docking cavity and a corresponding lateral tibial insert may be designed with the male extension that allows the lateral tibial insert to mate and slide together for a lock or engagement of the two pieces. The tibial insert lock mechanisms may include a variety of shapes, sizes and/or dimensions that may to obtain the best mechanical advantage for quick and strong locking of the medial and lateral tibial inserts and prevent mechanical failure of the joint.

In various embodiments, the modular tibial inserts may be designed with tibial insert mating and/or locking mechanisms, which can include a separate tibial insert locking tool or mating component that may be removable. For example, both the medial and lateral tibial inserts may be designed with female docking cavities, where the tibial lock tool slides within the medial and lateral docking cavities and mates the two pieces together for a frictional or other lock or engagement. The tibial insert lock mechanisms and the tibial locking tool may include a variety of shapes, sizes and/or dimensions that may to obtain the best mechanical advantage for quick and strong locking of the medial and lateral tibial inserts and prevent mechanical failure of the joint.

In various embodiments, the tibial locking tool may be designed with a tibial tray connector feature. The tibial locking tool with an integrated tibial tray connector can have dual functions—it may lock the medial and lateral tibial inserts together as well as lock the tibial insert assembly to the tibial tray. The tibial insert lock mechanism, the tibial locking tool and/or the tibial tray connector may include a variety of shapes, sizes and/or dimensions that may to obtain a mechanical advantage for quick and strong locking of the medial and lateral tibial inserts and/or the tibial tray to prevent mechanical failure of the joint.

In various embodiments, the modular tibial inserts with tibial insert friction lock mechanisms may be designed to attach to tibial trays with fixed-bearing features. In alternative embodiments, the modular tibial inserts with tibial insert friction lock mechanisms may be designed to attach to tibial trays with mobile-bearing features. Mobile-bearing features incorporated into a tibial tray design may facilitate more natural knee kinematics, in that the medial and lateral tibial inserts can be individually particularized while allowing the assembled insert to have multi-directional mobility, which may include rotation inside the tibial tray, anterior/posterior (A/P) movement, and/or medial/lateral (M/L) movement, and/or various combinations thereof.

In various embodiments, modular tibial inserts may be designed with a wide variety of alternative locking mechanisms, including adhesive and/or mechanical locking mechanisms or other fastening mechanisms rather than frictional locking mechanisms. These mechanical locking mechanisms may include a screw thread, a hinge-type design, clips, slide locking mechanisms, quick disconnect couplings, magnetic couplings, and/or any other locking or fastening mechanisms known in the art.

In various embodiments, the improved tibial implant component assembly may be suitable for use for total knee surgery or partial knee surgery, including multi-component systems incorporating tibial trays, tibial inserts, tools and methods. Alternatively, the tibial implant component assembly described herein may also be successfully applied to other damaged or diseased articulating joints, or opposing joint structures (i.e., creation of bone blocks and associated connective tissue anchoring locations on one or both opposing surfaces of a joint). Such joints can include various other joints of a body, e.g., ankle, foot, elbow, hand, wrist, shoulder, hip, spine or other joints.

BRIEF DESCRIPTION OF THE SEVERAL VIEWS OF THE DRAWINGS

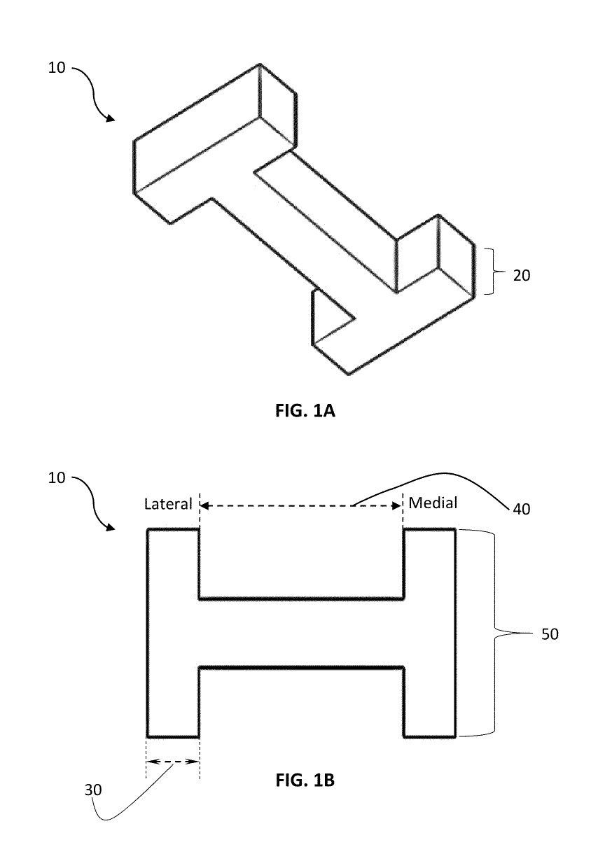

FIGS. 1A-1B depicts various exemplary views of one embodiment of a tibial locking tool formed in an “H” configuration;

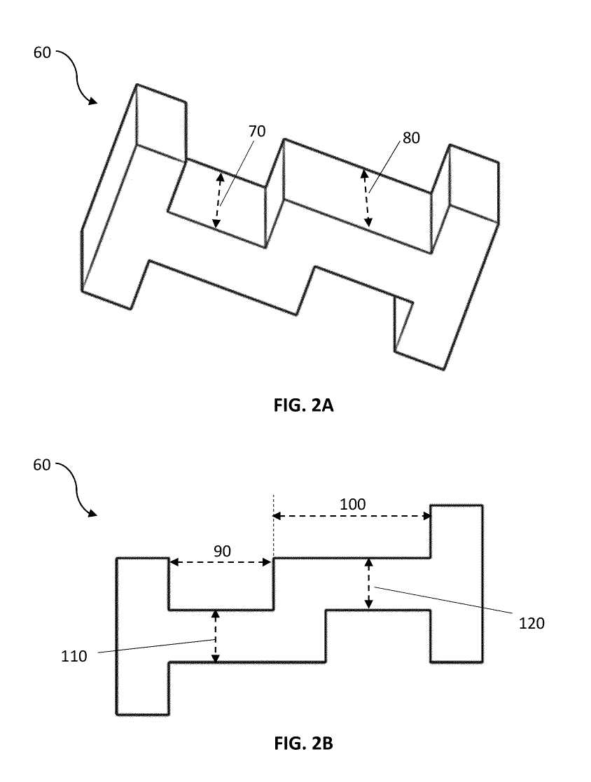

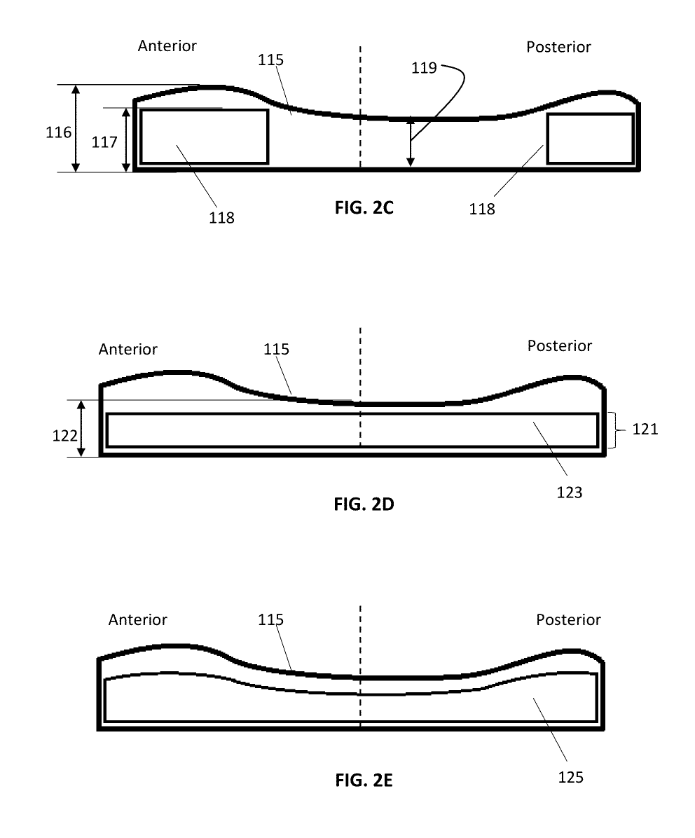

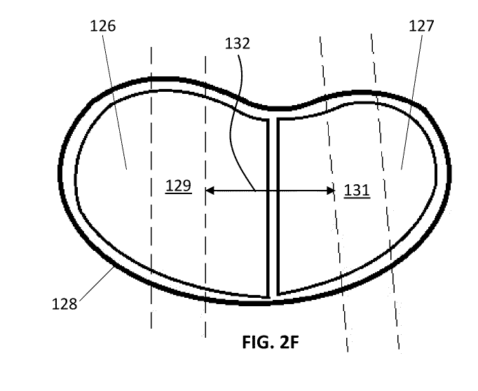

FIGS. 2A-2B depict various exemplary views of one alternative embodiment of a tibial locking tool in a stepped “H” configuration;

FIGS. 2C-2E depict various alternative tool designs and arrangements to accommodate variations in the thickness of an exemplary insert along an A/P direction;

FIG. 2F depicts a top plan view of a pair of tibial inserts and an associated tibial tray, with a pair of exemplary articulating zones;

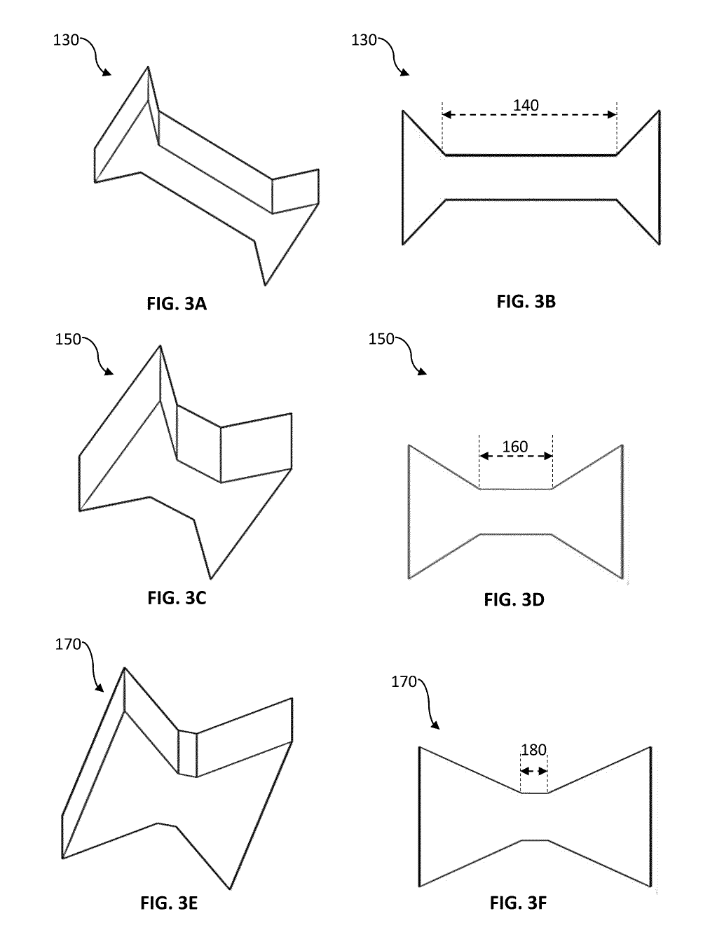

FIGS. 3A-3F depict various exemplary views of another alternative embodiment of a tibial locking tool;

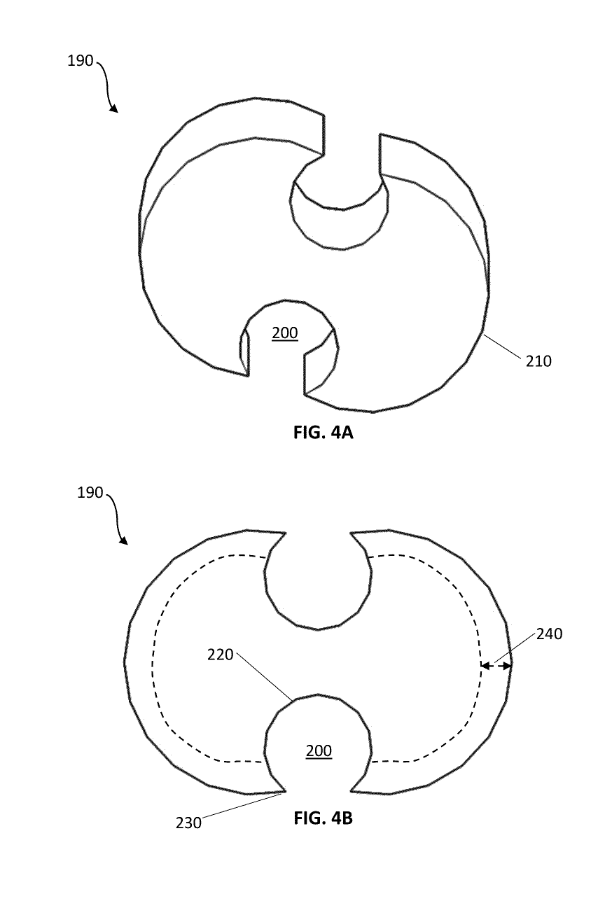

FIGS. 4A-4B depicts various exemplary views of another alternative embodiment of a tibial locking tool with a tibial tray connector in a dome-shaped configuration;

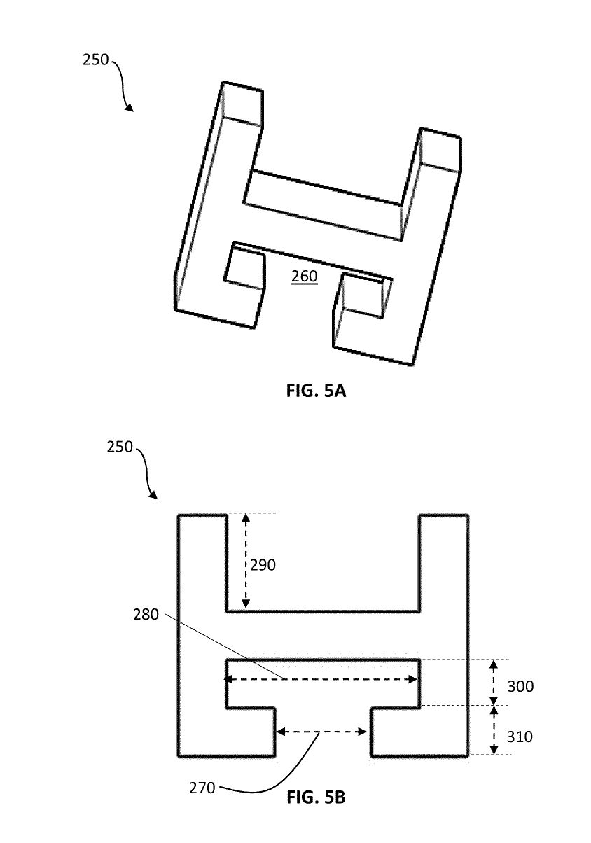

FIGS. 5A-5B depicts various exemplary views of another alternative embodiment of a tibial locking tool with a tibial tray connector in a “H” configuration;

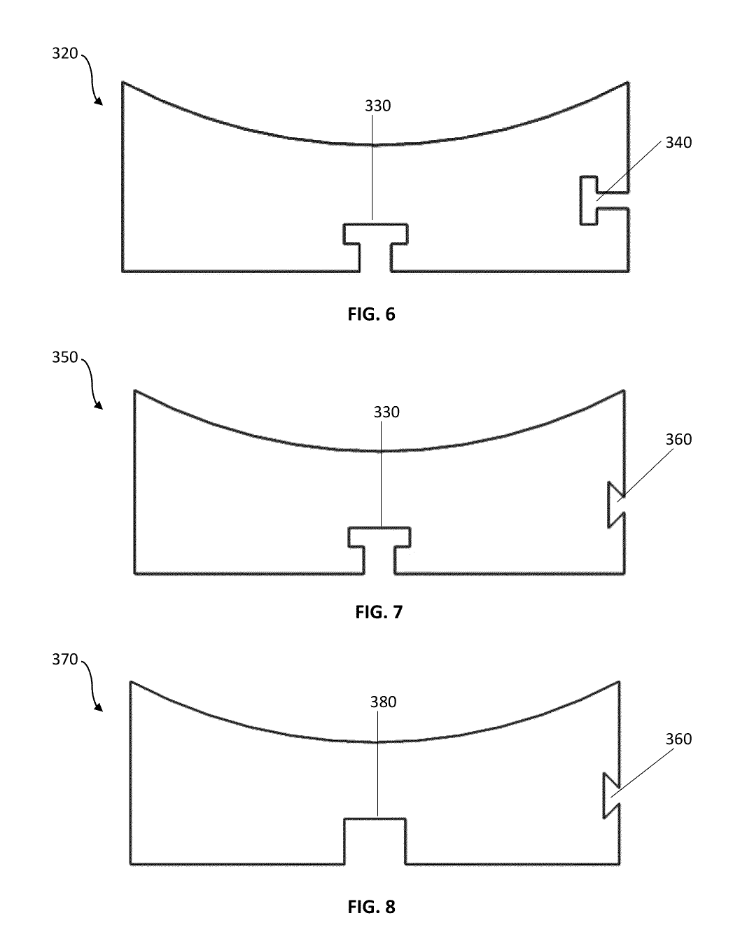

FIGS. 6-8 depict front views of various exemplary embodiments of a tibial insert with a tibial insert assembly locking mechanism and recessed attachments to a tibial tray;

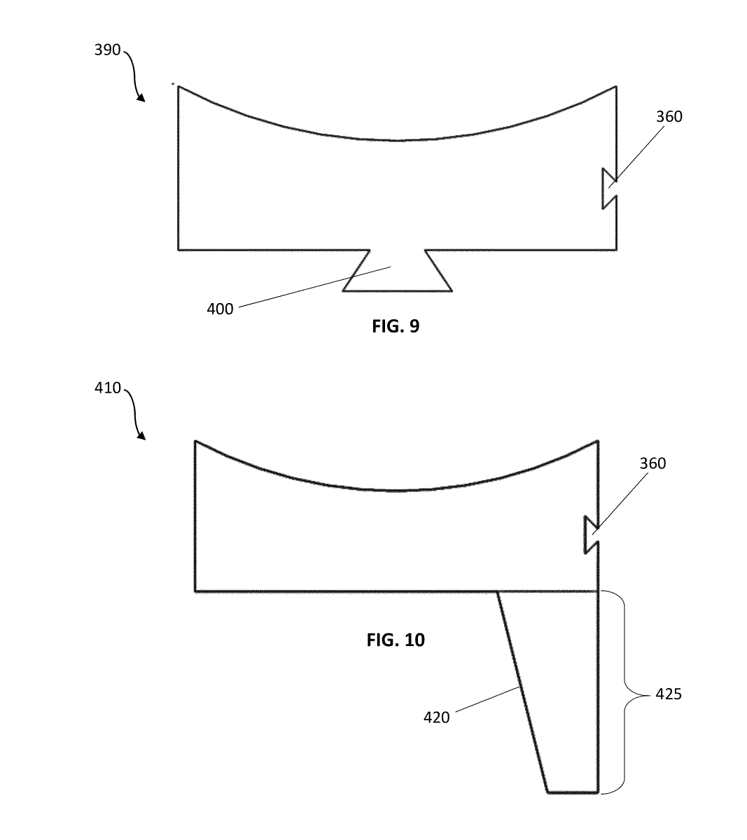

FIGS. 9-10 depict front views of various alternative embodiments of a tibial insert with a tibial insert assembly locking mechanism and post attachments to the tibial tray;

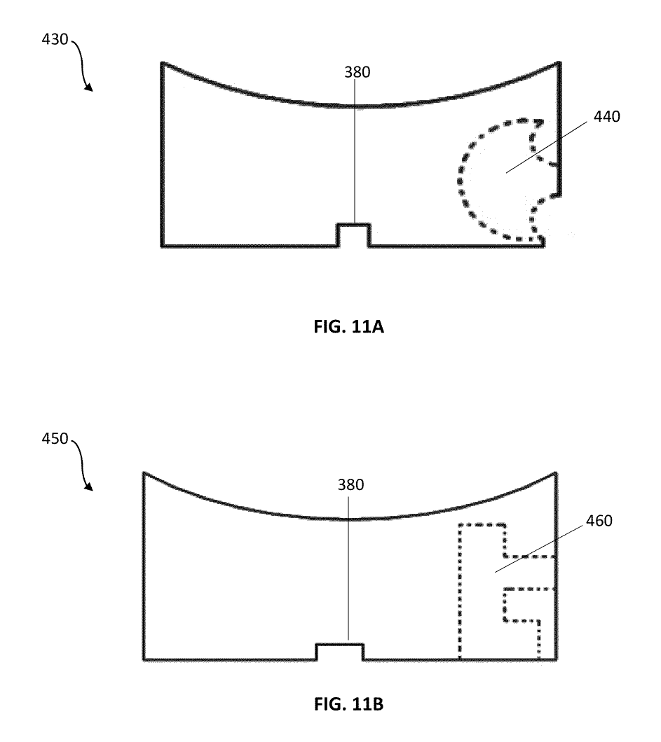

FIGS. 11A-11B depict front views of various alternative embodiments of a tibial insert with tibial insertion docking cavities and recessed constraint features;

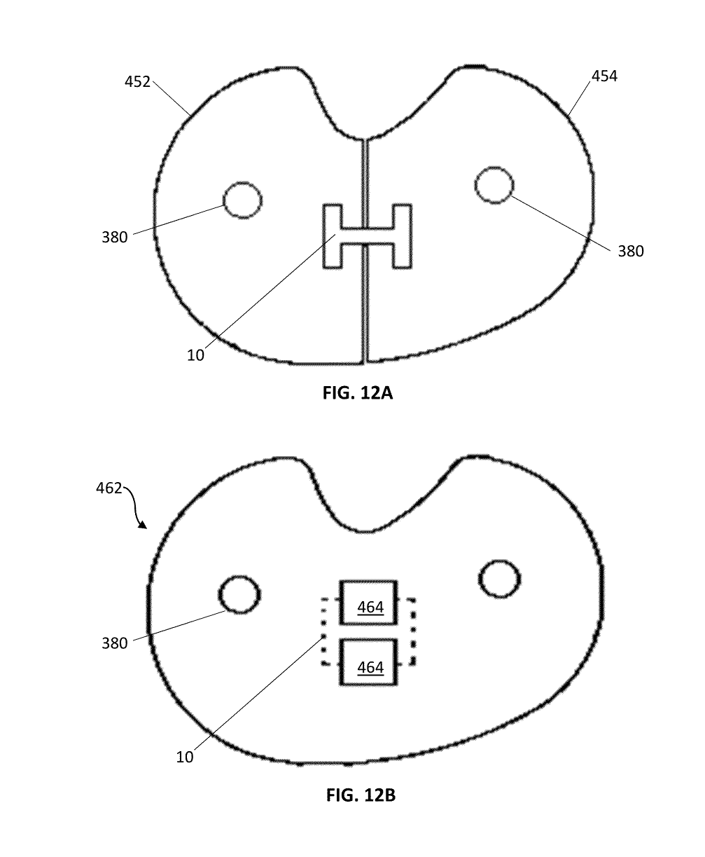

FIG. 12A depicts a bottom plan view of one embodiment of a tibial locking tool placed on a non-articulating (inferior) surface of the tibial insert assembly;

FIG. 12B depicts a top plan view of one embodiment of a tibial tray with rotational blocks;

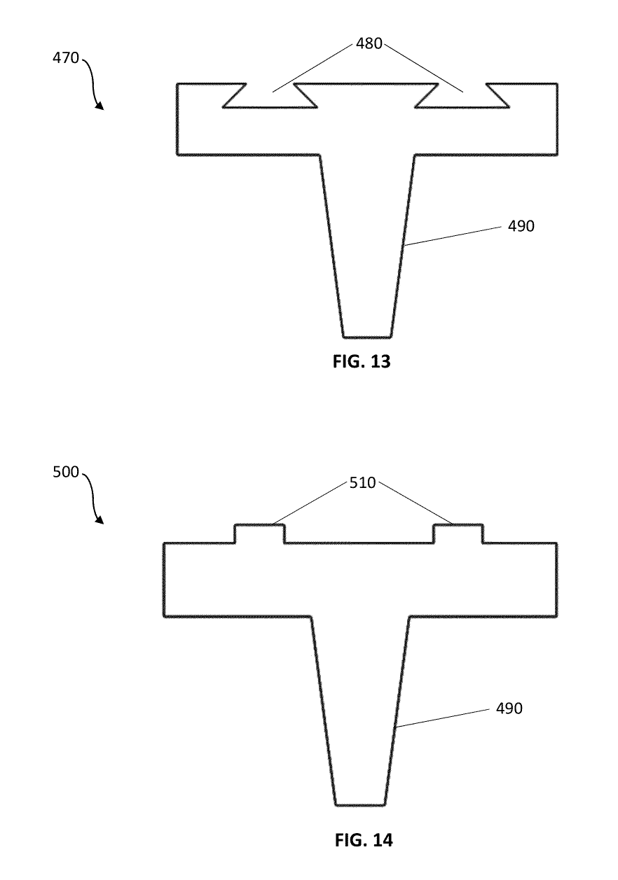

FIGS. 13-14 depict front views of various embodiments of a tibial tray with mobile-bearing features;

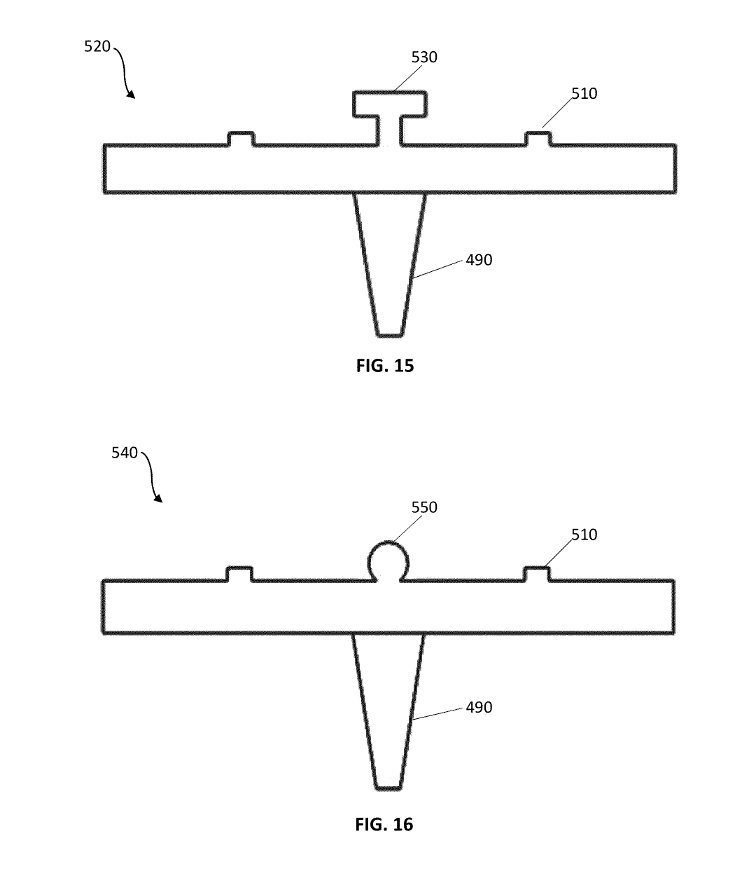

FIGS. 15-16 depict front views of various alternative embodiments of a tibial tray with mobile-bearing features and a tibial tray connector post;

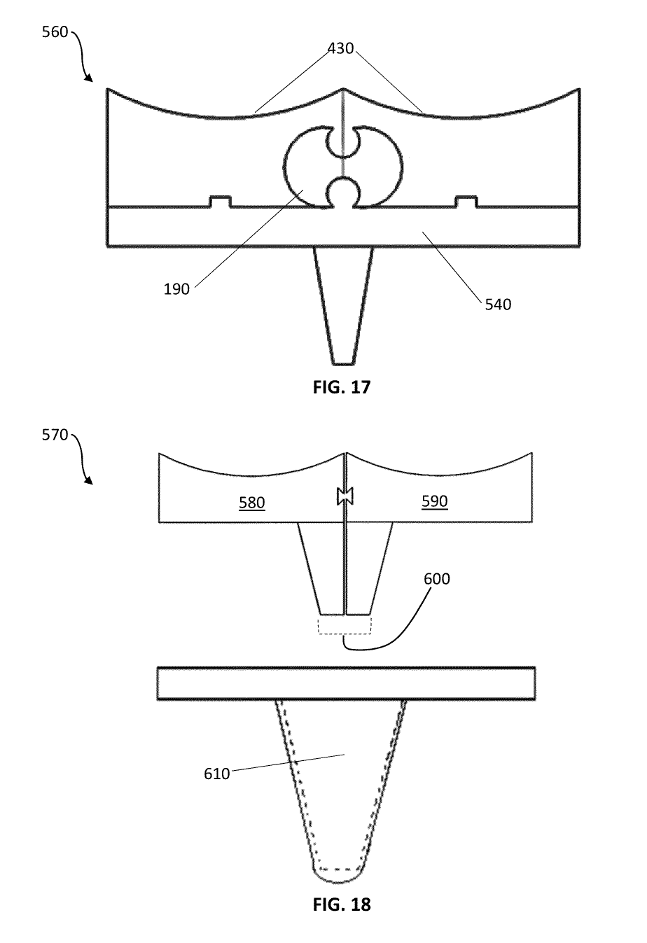

FIG. 17 depicts one embodiment of a frictional lock tibial component assembly;

FIG. 18 depicts an alternative embodiment of a frictional lock tibial component assembly with an inverted peg;

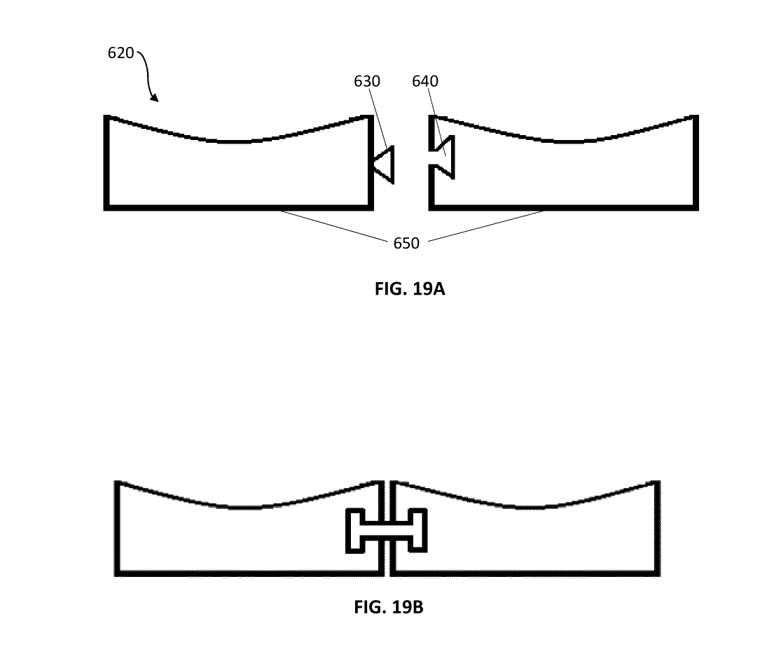

FIG. 19A depicts a front view of one embodiment of a tibial insert with an integrated dove-tail tibial insert friction lock mechanism;

FIG. 19B depicts a front view of another alternative embodiment of a tibial insert assembly with a mating or locking mechanism;

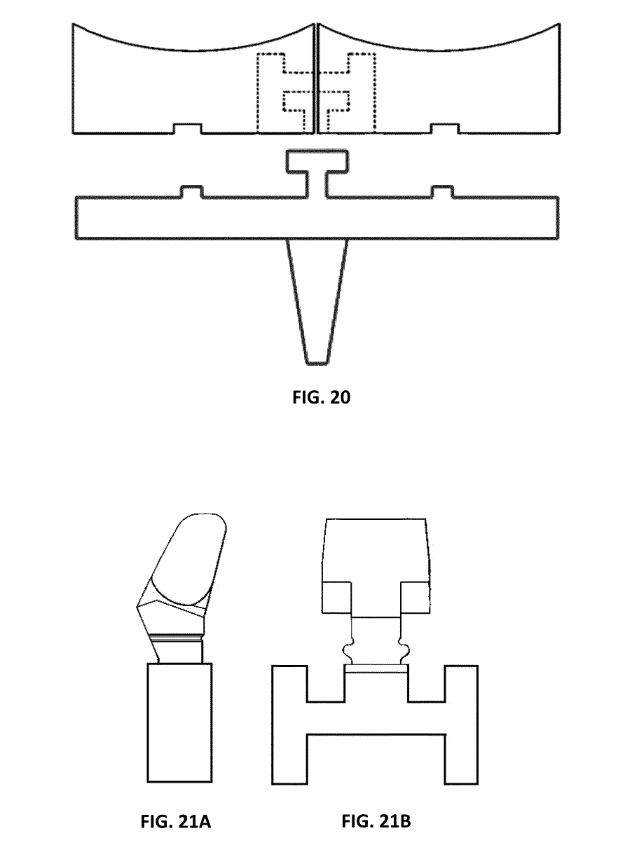

FIG. 20 depicts a Front view of an alternative embodiment of a tibial insert assembly with a mating or locking mechanism and integrated tibial tray connector;

FIGS. 21A and 21B depict the front and side view of a posterior-stabilized tibial locking tool; and

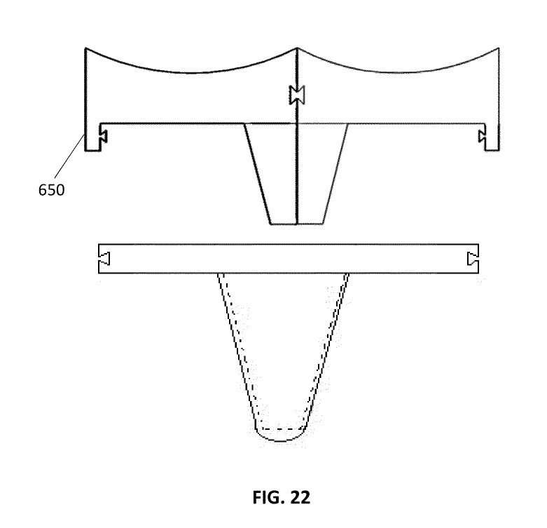

FIG. 22 depicts a front view of a tibial insert with an overhang dove-tail locking plate and a tibial tray with dove-tail cavity.

### DETAILED DESCRIPTION OF THE INVENTION

The drawings and the following description relate to preferred embodiments by way of illustration only. It should be noted that from the following description, alternative embodiments of the components and methods disclosed herein will be readily recognizable as viable alternatives that may be employed in one skilled in the art.

In one exemplary embodiment, the improved tibial implant component assembly may include a tibial locking tool, a medial tibial insert and a lateral tibial insert with a tibial insert locking mechanism, and a tibial tray with a tibial tray connector mechanism. In various embodiments the tibial tray may include fixed-bearing and/or mobile-bearing connection features for the assembled insert.

### Tibial Insert Selection and Mating

In various embodiments, a plurality of tibial inserts of different sizes, shapes, materials and/or other physical features (with some variations between each of the inserts) can be provided to a surgeon as part of an implant component kit for a surgical procedure. The tibial inserts can include different insert sets for each of the medial and lateral sections of a tibial tray, and the surgeon can select an appropriate combination of inserts (i.e., by “balancing” the knee by using the inserts or insert analogs such as “trial” components in a known manner) to create a desired implant design or knee motion and/or obtain some other desired outcome. Once appropriate inserts are selected, the surgeon may mate the selected medial and lateral tibial inserts together to create a tibial insert assembly, and then secure the insert assembly to the tibial tray in a single operation.

In one exemplary embodiments, the tibial inserts can include inserts having varying thicknesses, such as 2 mm, 4 mm, 6 mm and 8 mm. In various other embodiments, the inserts could comprise different materials, or differing surface convexities or other features, or differing articulation paths, etc.

### Tibial Locking Tools and Improved Tibial Inserts

In various embodiments, a tibial insert locking tool can be designed and/or selected to attach modular tibial inserts together. A tibial insert locking tool can provide the surgeon with the flexibility to select various medial or lateral tibial insert combinations for surgery, and then mate or connect the two tibial inserts prior to final attachment to the tray, thus, producing a single tibial insert assembly that is particularized to the patient's anatomy and/or the surgeon's desired repair. The medial or lateral tibial inserts may be designed with docking cavities or other features that allow the tibial locking tool to be connected, which could include placement from an anterior-posterior (A/P) direction and/or the inferior surface (non-articulating) surface of the modular tibial inserts. Once the surgeon connects the modular tibial inserts with the tibial locking tool, the surgeon may manipulate the tibial inserts as one rigid or substantially-rigid assembly. The surgeon may introduce this tibial insert assembly through a surgical incision (which could include a partially-open or limited surgical incision) to position onto the tibial tray. If desired, the surgeon may check the range of motion and joint stability to confirm that the knee is properly balanced. Should the surgeon observe undesired gaps, the surgeon may choose to remove the tibial insert assembly and remove the tibial locking tool to replace one or more of the medial or lateral tibial insert with another desired shape and/or size. The surgeon can then reconnect the two tibial inserts using the same tibial locking tool and reinsert the new rigid tibial assembly until a desired balancing result is observed. In various alternative embodiments, the surgeon may choose to remove the tibial locking tool from the inserts after the medial and lateral inserts are firmly attached to the tibial tray, or the tibial locking tool may remain as a permanent part of the implant.

Various embodiments of tibial locking tools may be provided in various shapes and sizes or configurations. FIG. 1A depicts an isometric view of one embodiment of an “H” configured tibial locking tool 10. This “H” configured locking tool 10 may be manufactured with a variety of depths 20 that may be designed to fit any depth and/or anterior/posterior or medial/lateral dimensions of a tibial insert if a surgeon so desires. In the exemplary embodiment, the locking tool can attach to the tibial inserts in an A/P direction. The depth 20 may be derived by substantially matching the depth of a tibial insert from the A/P direction, or it may be designed to have a depth that matches the highest thickness of the tibial insert or may have a lesser depth, and the “H” configured locking tool 10 can be slid into place.

In other embodiments, the depth of the “H” configured locking may be designed to have a curvature for easier insertion in the tibial inserts. The patellar tendon may interfere with tibial locking tools that have a straight anterior to posterior depth resulting in improper placement or inability to place the tibial locking tool within the tibial inserts. However, a tibial locking tool that may be designed with a curved anterior to posterior depth (not shown), the tibial locking tool can be inserted easier within the tibial inserts. Alternatively, the tibial locking tool may have a curvature in the medial and/or lateral direction (not shown) to accommodate any other obstruction and easy insertion.

In other embodiments, the “H” configured tibial locking tool may be inserted in an angular direction (not shown). For example, the “H” configured locking tool have medial and lateral side arms that are angled 20 degrees, where the anterior view will start from the medial side and the posterior view will end at the lateral side.

FIG. 1B depicts a front view of one embodiment of an “H” configured tibial locking tool 10. The “H” configured tibial locking tool 10 can be designed with a medial and lateral side arms. The height 50 and the width 30 of each medial and lateral side arms may be equal dimensions or it may be offset dimensions. Allowing for this flexibility allows the “H” configured tibial locking tool 10 to be adapted for use in either the medial or lateral tibial insert due to the change in the contours and non-planar surfaces when accompanying a standard tibial insert design known in the art. The height of the tibial locking tool medial and lateral arms may correspond to the minimum thickness of the tibial insert if desired. The length 40 of the “H” configured tibial locking tool 10 can also be designed with varying lengths. The length 40 design may accommodate a surgeon's desire to allow some amount of linear or rotational movement of the insert relative to the tray, which may be desirable for the patient. For example, a long length which allows for some spacing or gap between the medial and lateral tibial inserts may provide for increased mobility. Alternatively, where a short length is desired, leaving little or no gap between the medial and lateral tibial inserts, this may provide a stronger bond, but may also provide for decreased mobility. Furthermore, the length 40 may include considerations and/or design features that desirably reduce the opportunity for subsequent mechanical failure. Since the dimensions, shape and/or contour of a standard tibial tray can vary, the length 40 may accommodate placement in the thickest part of the tibial insert, if desired. Desirably, the manufacture may provide multiple lengths 40, depths 20, and/or heights of the tibial locking tool in a kit that may be available to the surgeon.

FIGS. 2A and 2B depict various exemplary views of an alternate embodiment of a tibial locking tool formed in a stepped “H” configuration 60. The lateral depth 70 and the medial depth 80 of the tool may be designed with equal dimensions or offset dimensions. Both the lateral depth 70 and the medial depth 80 may include a variety of depths designed to fit tibial tray designs known in the art.

In various embodiments, the depth of the tibial locking tool will also account for variation in the thickness of a given insert along the A/P direction. FIGS. 2C through 2E depict various tibial locking tool designs to accommodate any variation of the thickness of the tibial insert along an A/P direction. For example, FIG. 2C, depicts a side view of a tibial insert with multiple tibial locking tools positioned at the anterior and posterior walls. The tibial locking tool may have an arm thickness (medial and/or lateral arms) 117 less than a maximum thickness 116 of the tibial insert 115, but greater than a minimum thickness of the tibial insert. The design depicted includes a pair of such tibial locking tools 118, with each tool occupying the maximum thickness 116 of the tibial insert (i.e., an anterior and a posterior tibial tool).

FIG. 2D depicts a tibial locking tool 123 having an arm thickness 121 (medial and/or lateral) less than a minimum thickness 122 of the tibial insert 115. If desired, this tibial locking tool can extend along the entirety of the A/P length of the tibial insert 115, or may be provided in multiple sets (i.e., an anterior and a posterior tibial locking tool).

FIG. 2E depicts another alternative embodiment of a tibial locking tool 125, in which the tibial locking tool 125 includes a varying arm thickness that may follow the contour of the tibial insert 115. The tibial lock tool 125 may desirably corresponds to the thickness of the insert along the A/P direction. Because the inset may be somewhat compliant and/or flexible, the insertion of the tool can be accomplished relatively easily (depending upon the flexibility of the material), and when secured in a desired position may perform a self-locking feature.

FIG. 2F depicts a top plan view of a medial 127 and lateral 126 tibial inserts and associated tibial tray 128. This view depicts a pair of articulating zones, with one zone in each of the medial 129 and lateral 131 inserts. These zones desirably depict the intended zone of articulation upon which the femoral implant components articulates on the tibial inserts. In various embodiments, the locking or mating mechanism(s) between the medial 127 and lateral 126 inserts will desirably not extend into the articulating zones, but will rather be positioned within in the adjacent non-articulating zones 132 of the respective medial 127 and lateral inserts 126. In alternative embodiments, the locking or securement mechanism(s) may extend into the articulating zones, or may even extend fully through one or both of the medial 127 and lateral inserts 126, if desired. Furthermore, if the surgeon desires, the locking or mating mechanisms may extend into the non-articular zones on the peripheral portion of the tibial inserts, and/or the perimeter of the tibial inserts.

In various additional embodiments, a lateral length 90 and/or lateral height 110 of a stepped “H” locking tool 60, and the medial length 100 and the medial height 120 may be designed with equal dimensions or offset dimensions. As previously described herein, the medial height 120 and the lateral height 110 may correspond to the varying thickness of the medial 127 or lateral 126 tibial insert. In addition, the medial length 100 and the lateral length 90 may consider extension into articulating zones or within the non-articulating zones as described in FIG. 2F. This design might provide for additional flexibility in adapting the stepped “H” locking tool 60 to patient-specific tibial inserts, standard available tibial inserts or to mixed manufacturer tibial inserts.

FIGS. 3A-3E depict various alternate embodiments of a dove-tail configured tibial locking tool. The dove-tailed configured locking tool may be designed to accommodate long connections 130, a medium connection 150, and a short connection 170. The lengths as shown in 140, 160 and 180 may be derived from the surgeon's request or may be designed to allow for a desired amount of movement and rotation, as well as potentially maintaining a minimal mechanical advantage without subsequent failure of the connection. Furthermore, the angles (not shown) may vary depending on the amount of friction and/or retention forces desired between the connections.

The tibial locking tool may be adapted to have dual features—which in some embodiments may include a tibial insert frictional locking mechanism and a tibial tray connector. The tibial insert frictional locking mechanism feature may attach the medial and lateral tibial inserts together to create a rigid or substantially-rigid one-piece assembly, and the tibial tray connector feature may attach the one-piece assembly to the tibial tray. FIG. 4A depicts an isometric view of one exemplary embodiment of a dome-shaped tibial locking tool 190 with a tibial tray connector 200. The radius 210 of the dome-shaped tibial locking tool 190 may be provided in various dimensions. The shape of the radius 210 can give the dome-shaped tibial locking tool 190 additional strength by distributing the load carried along the radius, thereby spreading out the local effects of any load. The radius 210 can be optimized by selecting from a standard database of forces seen in the A/P and M/L direction, or it can be derived by having the patient undergoing various tests to obtain patient-specific measurements.

FIG. 4B depicts a front view of a dome-shaped tibial locking tool 190 with a tibial tray connector 200. The tibial tray connector 200 may also be dome-shaped 220. The tibial tray connector 200 may be adapted to provide little or no gap between the connector and the tray, leaving a sharp joint 230. The tibial tray connector 200 may also be designed to have some gap or other feature, providing for a neck (not shown), allowing the dome-shaped tibial locking tool to be adjusted centrally or closer to the articular surface of a tibial insert. Alternatively, the dome-shaped tibial locking tool 190 may include a rim 240 design, where the radius is a thin ring, instead of a dome shape.

The frictional tibial locking tool may have an integrated posterior stabilized (PS) post or cam as shown in FIG. 21A and 21B. The manufacturer may provide tibial locking tools with various posterior-stabilized (PS) cams or posts in a kit so that the surgeon could select the appropriate tibial locking tool that had the appropriate type of PS post to give the knee the appropriate amount of constraint. This would allow the surgeon to change the geometry of the post during the surgery and increase the constraint of the knee joint by selecting a post that fit more tightly in the box of the femoral component. It could be on articulating side—surrounding the periphery of the far medial and lateral edges.

FIGS. 5A and 5B depict various exemplary views of another alternate embodiment of an “H” configured tibial locking tool 250 with a captive peg tibial tray connector 260. The captive peg tibial connector 260 may be incorporated into the tibial locking tool to prevent dislocation by extrusion and by anterior lift-off during normal use of the knee. The captive peg tibial tray connector 260 may accommodate a variety of dimensions for the secure attachment to the tibial tray, such as the neck width 270 and neck height 310, and the lid width 280 and lid height 300. For example, the neck width 270 and lid height 300 may include a larger or thicker dimension to prevent long-term material fatigue or fatigue fracture when exposed to high or excessive stress levels. Alternatively, the tibial tray locking tool may be designed with various other configurations that can optimally attach the tibial inserts together and attach to the tray (see FIG. 20). The other configurations may resemble a dovetail, angular, dome, circular configurations for adaptation to the tibial locking tool.

In various embodiments, the modular tibial inserts may be designed with mechanical locking mechanisms or other fastening mechanisms rather than frictional locking mechanisms. These mechanisms may include one or more screw threads, hinge-type designs, clips, slide locking mechanisms, quick disconnect couplings, magnetic couplings, and/or any other locking or fastening mechanisms known in the art. For example, the mechanical connection may be a metal clip or other metal rod that could slide into a groove in each medial and lateral tibial insert (not shown). Alternatively, the mechanical connection may be a screw that would thread one tibial insert to the other tibial insert to connect the M/L tibial inserts together (not shown). Furthermore, the quick disconnect couplings may designed for low compression to easily mate the M/L tibial inserts and medium tension for one-handed operation to quickly disconnect the tibial inserts from each other.

In various embodiments, the improved tibial implant component assembly may include modified tibial inserts with docking cavities and mobile-bearing features. The mobile-bearing features on the tibial inserts may allow unconstrained movement, constrained movement (cone-in-cone, tibial tray posts, and/or stops) and/or constrained rotation movement (multi-directional cylindrical posts).

Creating mobile-bearing unconstrained features into the tibial inserts and/or tray features may be desirable. One example of a tibial insert 620 that incorporates a docking cavity for frictional locking is shown in FIG. 19. The tibial insert 620 has a dove-tail docking cavity 640, where the dove-tail post 630 easily slides into the dove-tailed shaped track to mate and lock the two tibial inserts together. In various embodiments, the non-articulating surface (or inferior surface) 650 of the tibial insert may be fixed to the tray by one or more peripheral edges (not shown), or alternatively could be attached to the tibial tray for unrestrained and/or partially-restrained movement of the tibial inserts relative to the tray (not shown). The inferior surface 650 of the tibial inserts may be allowed to slide freely on a rimless, flat and polished tibial tray surface (not shown), or may include some limitation on the movement and/or rotation of the tibial inserts. If fully unconstrained, the implant could rely on the conformity of the tibial inserts to the femoral component and the tension of the soft tissues for stability of the construct.

Alternatively, more controlled mobility of the tibial inserts may be desired, and such designs may incorporate various constraint features into the tibial inserts that can accommodate tibial trays that have captive peg recesses, non-captive peg recesses, inverted peg posts and dove-tail posts, and/or various combinations thereof. FIGS. 6 through 8 depict views of various embodiments of a tibial insert with various tibial insert docking cavity configurations and various recessed constraint features to accommodate a tibial tray with captive peg recesses and non-captive peg recesses.

FIG. 6 shows a tibial insert 320, where the recessed constraint feature is a captive peg recess 330 and a captive peg frictional docking cavity 340. As described herein, the captive peg configuration may help prevent dislocation by extrusion and by anterior lift off. FIG. 7 shows a tibial insert 350, where the recessed constraint feature is a captive peg recess 330 and a dove-tail docking cavity 360. FIG. 8 shows a tibial insert 370, where the recessed constraint feature is a non-captive peg recess 380 and a dove-tail docking cavity 360. The non-captive peg recess 380 can accommodate a tibial tray that may include at least two cylindrical pegs arising from the superior surface of the tibial tray. The use of cylindrical non-captive recesses 380 may help control the anterioposterior, mediolateral, and rotary mobility of the tibial inserts. These are only illustrations of the potential combinations of docking cavity configurations and the various recessed constraint features that may be known in the art. All the variations of the tibial locking tool configurations described herein may also be incorporated into a docking cavity.

In other embodiments, the tibial insert may be designed with a tibial tray overhang locking plate 650 as shown in FIG. 22. The overhang locking plate 650 may overhang from the perimeter edge of the tibial insert and contains dove-tail extension to mate and lock into the tibial tray. The tibial tray may also be modified to include a dove-tail shaped cavity or other configuration that may produce a mechanical advantage. This overhanging locking plate 650 may offer additional medial/lateral and anterior/posterior constraint between the tibial insert and the tibial tray. The locking mechanism include a variety of other configurations, or mechanical fastening mechanisms as known in the industry

Furthermore, FIGS. 9 and 10 depict views of various embodiments of a tibial insert with various tibial insert docking cavity configurations and various post constraint features to accommodate a tibial tray with a dove-tail track and an inverted peg cone. FIG. 9 shows a tibial insert 390 that incorporates a post constraint feature that is a dove-tail post 400 and has a dove-tail docking cavity 360. FIG. 10 shows a tibial insert 410 that incorporates a post constraint feature that is an inverted peg 420 and has a dove-tail docking cavity 360. A tibial insert 410 with an inverted peg 420 constraint feature may help in preventing dislocation by extrusion because the inverted peg 420 may be designed in longer extension heights. The inverted peg 420 has a tapering extension 425 that sits inside the stem of the tibial tray (see FIG. 18) and may not be limited in height by the thickness of the tibial insert, if desired. The tapering extension 425 may be designed long enough to protect the tibial insert against dislocation by extrusion.

FIG. 11A depicts a front view of one alternative embodiment of a tibial insert 430 with a non-captive recessed constraint feature 380 and dome-shaped frictional docking cavity 440 that can be capable of accommodating a dome-shaped tibial locking tool 190 as shown in FIG. 4B. FIG. 11B depicts the front view of one embodiment of a tibial insert 450 with a non-captive recessed constraint feature 380 and “H” configured captive peg frictional docking cavity 460 that will accommodate an “H” configured tibial locking tool 250 as shown in FIG. 5B.

FIG. 12A depicts a bottom view of one embodiment of an “H” configured frictional locking tool 10 placed on the non-articulating (inferior) surface of both the lateral tibial insert 452 and the medial tibial insert 454. The surgeon may prefer that the frictional locking tools are placed on the non-articulating (inferior) surface of the both the medial 454 and lateral 452 inserts to prevent any soft tissue from potentially contacting the “H” configured frictional locking tool 10. The depth of the “H” configured frictional locking tool 10 may be controlled by the maximum thickness of either the lateral tibial insert 452 and/or the medial tibial insert 454. Furthermore, the “H” configured frictional locking tool 10 may be positioned anywhere along the connection axis to accommodate various recessed or post constraint features as described herein

In other embodiments, the “H” configured frictional locking tool 10 may be positioned flush with the non-articulating inferior surface of the tibial inserts to allow recessed constraint features and/or post constraint features to be incorporated. Alternatively, the “H” configured frictional locking tool 10 may also be positioned where at least a portion thereof extends from the non-articulating inferior surface (not shown). The at least a portion of the “H” configured frictional locking tool 10 that extends outward of the inserts may be designed to secure and/or mate into an improved tibial tray 462 with rotational blocks 464 as shown a top view of FIG. 12B. The tibial tray 462 may include recessed cylindrical constraint features 380 or may be flat using only the rotational blocks 464 to constrain movement of the tibial inserts.

In another embodiment, the H configured tibial locking tool 10 and other tibial locking tool configurations may have a feature that engages an anti-release mechanism on the tibial tray (not shown) such that the anti-release mechanism on the tibial tray prevents and/or limits the movement of the tibial locking tools after they have been locked into the tibial inserts. For example, the H configured tibial locking may include a recessed feature where the anti-release mechanism on the tibial tray can mate with the recessed feature and prevent and/or limit movement.

The tibial inserts and the tibial locking tools may be manufactured using standard plastic materials. For example, a polymer such as ultrahigh molecular weight polyethylene may be used. If ultrahigh molecular weight polyethylene is used, it may be desirable to use a cross-linked form of this material. Use of such a polymer should be advantageous in that the posts, extensions, tabs, and/or mechanical connections of the tibial inserts may be flexible for rigid assembly of the tibial insert and the tibial tray. Alternatively, the manufacturer may consider the tibial locking tool may be manufactured from metal or some metal alloy. Should the configuration of the tibial locking tool require the rigidity and the strength, it may be desirous to combine a plastic tibial insert with a metal tibial locking tool.

### Improved Tibial Trays

The improved tibial implant component assembly that may include a tibial tray with a tibial tray connector mechanism and optional mobile-bearing features. A mobile-bearing insert assembly can be created to alleviate any perceived disadvantages in a comparable fixed-bearing bearing insert assembly. For young, active patients undergoing significant activity or carrying extra weight, a fixed-bearing insert may wear more quickly and eventually fail or loosening, resulting in instability, pain and eventually joint failure. In contrast, mobile-bearing inserts can rotate short distances within the metal tibial tray, desirably allowing the implant to more closely mimic the function of the human meniscus by accommodating the natural combination of rolling and sliding movements of the knee. In such a case, the tibiofemoral relative motion could occur in two places, with the first motion occurring between the inferior surface of the tibial insert assembly and the tibial tray (since the inferior surface is not rigidly fixed to the tibial tray), and the second motion accommodated between the articulating surface of the tibial insert assembly and the femoral component. The underlying dual motion in a mobile-bearing insert assembly design would desirably not restrict the natural movement of the femoral component as much as a comparable fixed-bearing design, thereby providing for increased congruency between the two components and uncoupling some of the articulation forces at the prosthesis-bone interface. This congruency and the uncoupling would desirably lead to an increased contact area, lower contact stresses and/or reduced wear in comparison to a corresponding fixed tibial insert assembly design.

In one embodiment, the mobile-bearing feature on a tibial tray may allow unconstrained tibial insert movement. The inferior surface (non-articulating surface) of the medial and lateral tibial inserts may have planar surfaces that may be allowed to slide freely on a rimless, flat and polished tibial tray surface (not shown). This unconstrained tibial tray (not shown) may not limit the movement of the tibial inserts to a significant degree, but might rather rely on the conformity of the tibial inserts to the femoral component and the tension of the soft tissues. If desired, the manufacturer may incorporate various constraint features onto the tibial tray for more controlled mobility of the tibial inserts, such as tibial trays that have captive peg posts, non-captive peg posts, inverted peg posts and dove-tail posts, and/or various combinations thereof.

In one preferred embodiment, the tibial trays may incorporate various mobile-bearing constraint features, such as those shown in FIGS. 13 and 14. FIGS. 13 and 14 depict views of various embodiments of a tibial tray with mobile features having a dove-tail track 480 and non-captive cylindrical posts 510. The tibial insert dove-tail post 400 (as shown in FIG. 9) can slide neatly within the tibial tray dove-tail track 480 (as shown in FIG. 13), which may control the movement of the tibial inserts by keeping the tibial inserts on a track governed by the selected configuration. This type of design may help eliminate dislocation by anterior lift off of the tibial insert. Alternatively, the non-captive peg recess 380 (as shown in FIG. 8) can be positioned onto the non-captive cylindrical posts 510, where non-captive cylindrical posts 510 can be used to control and/or limit the anteroposterior, mediolateral and rotary mobility of the tibial inserts. Furthermore, the tibial trays may also be designed with distally extending stems 490 and keels (not shown) as known in the art. It may be desirous to design tibial trays fixed features to lock the tibial inserts with tibial frictional lock mechanisms.

In various embodiments, the locking mechanism of the tray and/or insert assembly will desirably be designed to secure the fully assembled insert assembly, which may include features substantially less complex and/or more robust than locking arrangements currently used to secure each of the medial and lateral insert components individually. Such an arrangement has the potential to significantly improve the locking mechanism incorporated on the tibial tray, which in various embodiments may function differently for the single tibial insert assembly as compared to separated modular tibial inserts. Traditionally, a total knee replacement system would require that the surgeon fixes the individual medial and lateral tibial inserts using tray locking mechanisms during surgery. The surgeon would maneuver, position and engage a first tibial insert to the tibial tray, and then would repeat this operation for the second tibial insert. Aside from the repeating of the effort required for insertion and locking of two inserts to the tray, the duplicative locking mechanisms add additional complexity into the tray design (thereby significantly increasing manufacturing cost) and can significantly contribute to the chance of implant failure and/or surgeon error, in that the chance that one locking mechanism may fail has been doubled, as well as doubling the chance that an insert does not fully seat into a respective locking mechanism. Improper placement may lead to dislodgement or dislocation of the tibial insert from the tibial tray, or can lead to varying degrees of motion between the tibial insert and the tibial tray (which could at best result in undesirable undersurface wear and the production of polyethylene particles, and at worst result in failure of the implant and the need for subsequent surgery).

In contrast, the methods of the current invention allow the surgeon to properly attach the tibial insert assembly in a single operation. Moreover, the single, larger insert assembly of the present invention may be easier for the surgeon to grasp and/or manipulate, as compared to the smaller individual inserts. In addition, the attachment between the medial and lateral insert components can significantly increase the rigidity and/or securement of the individual medial and lateral components, significantly reducing the opportunity for movement between the insert and the tray (for fixed-bearing designs) as well as reducing the complexity of fixation and/or potential for unwanted motion (for mobile-bearing designs). Reduction of insert motion (in fixed-bearing designs) and/or reduction of unwanted insert motion (for mobile-bearing designs) can significantly reduce the production of particle debris and/or alter particle debris size, thereby reducing potential causes of osteolysis and implant failure (i.e., by triggering an autoimmune reaction causing resorption of living bone).

The improved tibial trays may include mobile features and tibial tray connector posts that attach to tibial tray connectors on a tibial locking tool. FIGS. 15 and 16 depict front views of various embodiments of a tibial tray with mobile-bearing features and tibial tray connector posts in a captive post 530 configuration, and a spherical post 550 configuration. Each of these trays may be used in combination of the various constrained and unconstrained mobile features described herein to provide for locking of the tibial inserts with tibial locking tools to the tibial tray.

Alternatively, the tibial inserts with tibial locking tools may be attached to tibial trays with fixed-bearing features. The fixed-bearing tibial trays (not shown) may include sidewalls, projections, flanges or centrally located dovetails that mate with features on the tibial inserts to lock the tibial tray and tibial inserts together (or various combinations of one or more such locking arrangements). The features incorporated into fixed-bearing tibial tray design may be advantageous for a surgeon when compared to mobile-bearing design. For example, mobile bearing prostheses typically require that balancing of the knee be accurate. Since the knee stability depends on well-balanced ligaments and soft tissues around the new knee joint, the movement allowed in such mobile bearing prosthesis may make it more difficult to accurately balance leading to dislocation of the mobile bearing prosthesis. Another example could include the wear inherent in mobile bearing prostheses—the upper surface of the insert assembly in contact with the femoral components and the lower surface in contact with the tibial component may experience uneven wearing of the surfaces. In some cases, a higher incidence of burnishing, pitting/third-body embedding, and/or scratching wear patterns on the lower surface than compared to the higher surface may be experienced, which may lead to increased particulate debris, significant alteration of knee motion and/or loading and/or subsequent implant failure. Many of the issues observed in a mobile-bearing tibial tray may be resolved should the surgeon decide to implant a fixed-bearing tibial tray with features of the present invention.

FIG. 17 depicts a front view of one embodiment of a frictional lock tibial component assembly 560 using a dome-shaped tibial locking tool 190. The frictional lock tibial component assembly 560 highlights the ability to lock the tibial inserts 430 with the dome-shaped tibial locking tool 190 to the tibial tray 540. FIG. 18 depicts an alternative embodiment of an inverted peg frictional lock tibial component assembly 570. The inverted peg lateral insert 480 and the inverted peg medial insert 590 may be mated together and attached with a short dove-tail tibial insert locking tool 170 (such as the design shown in FIG. 3E). However, other dove-tail tibial insert lock tool lengths may be used. Once the inverted peg lateral insert 480 and the inverted peg medial insert 590 are mated, the mating produces a uniform inverted peg 600 that will fit inside a hollow stem 610 of the tibial tray.

It will be appreciated that the improved tibial component system (i.e., the tibial tray, tibial inserts and tibial locking took) could include several sizes and/or shapes of each of the components. Different sizes or shapes of tibial inserts may be used interchangeably on a single tibial tray. Thus, the tibial insert that provides the optimum contact surface for the femoral component can be selected to be attached to either fixed and mobile tibial trays. Further, the tibial locking tool configurations may match the configurations of the tibial tray connector posts, and/or the configurations may not necessarily match the same configuration (i.e., combinations of configurations may be used). For example, a tibial insert with a dove-tail docking cavity does not have to be designed to connect with a dove-tail tibial tray connector. The tibial tray may incorporate other tibial tray locking mechanisms as contemplated herein or as standard in the industry In various embodiments, concerns associated with reduced contact areas, edge loading, contact stress and polyethylene wear from mismatched femoral components and fixed tibial inserts bearings could be reduced or eliminated by using a mobile bearing tray design, if desired.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the invention.

1. A tibial insert assembly for use during treatment of a knee joint of a patient, the insert assembly comprising:
a first tibial insert having an upper articulating first surface, a lower first surface, and a peripheral first surface extending between the upper first articulating and lower first surfaces;
a second tibial insert having an upper articulating second surface, a lower second surface, and a peripheral second surface extending between the upper second articulating and lower second surfaces;
the peripheral first surface including a first locking mechanism component;
the peripheral second surface having a second locking mechanism component;
the first and second locking mechanism components operable to rigidly secure the first and second tibial inserts together.

2. The tibial insert assembly of claim 1, wherein at least one of the first or second tibial inserts includes an engagement mechanism for engaging a tibial tray.

3. The tibial insert of claim 2, wherein the engagement mechanism inhibits rotational movement of the tibial insert assembly relative to the tibial tray.

4. The tibial insert of claim 2, wherein the engagement mechanism inhibits translational movement of the tibial insert assembly relative to the tibial tray.

5. A tibial insert assembly for use during treatment of a knee joint of a patient, the insert assembly comprising:
a first tibial insert having an upper articulating first surface, a lower first surface, and a peripheral first surface extending between the upper first articulating and lower first surfaces;
a second tibial insert having an upper articulating second surface, a lower second surface, and a peripheral second surface extending between the upper second articulating and lower second surfaces;
a locking tool component;
the peripheral first surface including a first opening for accommodating a first portion of the locking tool component;
the peripheral second surface including a second opening for accommodating a second portion of the locking tool component;
wherein when the first and second portions of the locking tool component are positioned in the first and second openings, the first and second inserts are rigidly secured together.

6. The tibial insert of claim 5, wherein the locking tool component further includes an engagement mechanism for attaching to a tibial tray.

7. The tibial insert of claim 6, wherein the engagement mechanism inhibits rotational movement of the tibial insert assembly relative to the tibial tray.

8. The tibial insert of claim 6, wherein the engagement mechanism inhibits translational movement of the tibial insert assembly relative to the tibial tray.
